(19) United States
(12) Patent Application Publication
(10) Pub. No.: US 2006/0275756 A1
(43) Pub. Date: Dec. 7, 2006

(54) Displacement assay for detecting ligate-ligand association events

(76) Inventor: Gerhard Hartwich, Munchen (DE)

Correspondence Address: CROCKETT & CROCKETT, 24012 CALLE DE LA PLATA, SUITE 400, LAGUNA HILLS, CA, 92653, US

(21) Appl. No.: 10/487,815

(22) PCT Filed: Apr. 6, 2002
(86) PCT No.: PCT/DE02/01269

§ 371 Date: Feb. 25, 2004

(30) Foreign Application Priority Data
Aug. 25, 2001 (DE) 101416911

(51) Int. Cl. C12Q 1/68; G01N 33/53; C07K 16/46; C07H 21/04

(52) U.S. Cl. 435/6; 435/7.1; 530/391.1; 536/25.32

(57) ABSTRACT

Described is a method for detection of ligate-ligand association events, the method comprising the steps: providing a modified surface, the modification consisting in the attachment of at least one type of ligate; providing signal ligands; providing a sample having ligands; bringing a defined quantity of the signal ligands into contact with the modified surface and bringing the sample into contact with the modified surface; detecting the signal ligands; and comparing with reference values the values obtained from the detection of the signal ligands.

### FIELD OF THE INVENTION

The present invention is directed to a method for detection of ligate-ligand association events.

### BACKGROUND OF THE INVENTION

Immunoassays and, increasingly, also DNA and RNA sequence analysis are being employed in disease diagnosis, toxicological test procedures, genetic research and development, and in the agricultural and pharmaceutical sectors. In addition to the known serial methods using autoradiographical or optical detection, increasingly, parallel detection methods by means of array technology using what are known as DNA or protein chips are being applied. For the parallel methods, too, the actual detection is based on either optical, radiographical, mass spectrometric or electrochemical methods.

In addition to their applications for sequencing, oligonucleotide or DNA chips can also be used for SNP (single nucleotide polymorphism) or gene expression analysis, since they allow the activity level of a large number of individual active genes (cDNA or mRNA) of a specific cel type or tissue to be measured in parallel, which is possible with conventional (serial) gene detection methods only with difficulty or at great expense. The analysis of pathologically modified gene activities, in turn, can contribute to clarification of disease mechanisms and identification of new points of attack for therapeutic application. In addition, (only) DNA chips allow so-called pharmacogenomic studies during clinical development, which can significantly increase the effectiveness and safety of the drugs. The pharmacogenomic studies focus on the question of which genetic factors are responsible for patients displaying differing reactions to the same drug. Extensive polymorphism analyses (base-pair mismatch analyses) of genes that encode important metabolic enzymes can uncover answers to such questions.

To analyze genes on a chip, a library of known DNA sequences (“probe oligonucleotides”) is attached to a surface in an ordered grid such that the position of each individual DNA sequence is known. If fragments of active genes (“target oligonucleotides”) whose sequences are complementary to specific probe oligonucleotides on the chip exist in the test solution, the target oligonucleotides can be identified (read) by detecting the appropriate hybridization events on the chip.

Protein chips whose test sites carry specific antigen (or antibody) probes instead of probe oligonucleotides can be employed in proteome analysis or in parallelization of diagnostics.

The use of radioactive labels in DNA/RNA sequencing is associated with several disadvantages, such as elaborate, legally required safety precautions in dealing with radioactive materials. For fluorescence and mass spectrometric detection, the cost of equipment is very high.

Some of the disadvantages of labeling with radioactive elements or fluorescent dyes can be avoided if association events are detected based on the associated change in the electrochemical properties (cf. WO 97/46568, WO 99/51778, WO 00/31101, WO 00/42217).

For both protein analysis and DNA analysis, it is desirable and, for the user, advantageous when the targets (antibody/antigen or DNA fragment) need not be modified with a detection label.

Thus, although there are many options for detecting ligate-ligand associates, there is great need for simple, economical, and reliable detection principles that can be carried out easily, especially in the area of lower-density arrays (low-density DNA and protein chips with few to a few hundred test sites per cm², e.g. for so-called POC (point-of-care) systems).

### DESCRIPTION OF THE INVENTION

Therefore, it is the object of the present invention to create for detection of ligate-ligand associations a method that does not exhibit the disadvantages of the background art.

This object is solved by the method according to independent claim 1 and the kit according to independent claim 37. Further advantageous details, aspects and designs of the present invention are evident from the dependent claims, the description, the drawings and the examples.

The following abbreviations and terms will be used in the context of the present invention:
DNA deoxyribonucleic acid
RNA ribonucleic acid
PNA peptide nucleic acid (synthetic DNA or RNA in which the sugar-phosphate moiety is replaced by an amino acid. If the sugar-phosphate moiety is replaced by the —NH—(CH₂)₂—N(COCH₂-base)-CH₂CO— moiety, PNA will hybridize with DNA.)
A adenine
G guanine
C cytosine
T thymine
U uracil
base A, G, T, C or U
bp base pair
nucleic acid At least two covalently-joined nucleotides or at least two covalently-joined pyrimidine (e.g. cytosine, thymine or uracil) or purine bases (e.g. adenine or guanine). The term nucleic acid refers to any backbone of the covalently-joined pyrimidine or purine bases, such as the sugar-phosphate backbone of DNA, cDNA or RNA, a peptide backbone of PNA, or analogous structures (e.g. a phosphoramide, thiophosphate or dithiophosphate backbone). An essential feature of a nucleic acid within the meaning of the present invention is that it can sequence-specifically bind naturally occurring cDNA or RNA.
nt nucleotide
nucleic acid oligomer Nucleic acid of a base length that is not further specified (e.g. nucleic acid octamer: a nucleic acid having any backbone in which eight pyrimidine or purine bases are covalently bound to each other).
na oligomer nucleic acid oligomer
oligomer Equivalent to nucleic acid oligomer.
oligonucleotide Equivalent to oligomer or nucleic acid oligomer, e.g. a DNA, PNA or RNA fragment of a base length that is not further specified.
oligo Abbreviation for oligonucleotide.
mismatch To form the Watson-Crick double-stranded nucleic acid oligomer structure, the two single strands hybridize in such a way that the base A (or C) of one strand forms hydrogen bonds with the base T (or G) of the other strand (in RNA, T is replaced by uracil). Any other base pairing does not form hydrogen bonds, distorts the structure and is referred to as a “mismatch”.
ss single strand
ds double strand
substrate, cofactor, Complex binding partner of a protein (of an enzyme). coenzyme
antibody Complex binding partner of an antigen.
antigen Complex binding partner of an antibody.
receptor Complex binding partner of a hormone.
hormone Complex binding partner of a receptor.
oxidizing agent A chemical compound (chemical substance) that oxidizes another chemical compound (chemical substance) by taking up electrons from this other chemical compound (chemical substance).
reducing agent A chemical compound (chemical substance) that reduces another chemical compound (chemical substance) by giving up electrons to this other chemical compound (chemical substance).
redox-active Refers to the property of a moiety of giving up electrons to a suitable oxidizing agent or taking up electrons from a suitable reducing agent under certain external conditions.
EDTA ethylenediamine tetraacetate (sodium salt)
sulfo-NHS N-hydroxysulfosuccinimide
NHS N-hydroxysuccinimide
EDC (3-dimethylaminopropyl)-carbodiimide
HEPES N-[2-hydroxyethyl]piperazine-N′-[2-ethanesulfonic acid]
Tris trishydroxymethylamino methane
ligand Refers to molecules that are specifically bound by ligates. Examples of ligands within the meaning of the present invention are substrates, cofactors or coenzymes of a protein (of an enzyme), antibodies (as the ligand of an antigen), antigens (as the ligand of an antibody), receptors (as the ligand of a hormone), hormones (as the ligand of a receptor) or nucleic acid oligomers (as the ligand of the complementary nucleic acid oligomer).
ligate Refers to a (macro-)molecule on which are located specific recognition and binding sites for the formation of a complex with a ligand (template).
linker A molecular link between two molecules or between a surface atom, surface molecule or surface molecule group and another molecule. Linkers can usually be purchased in the form of alkyl, alkenyl, alkynyl, heteroalkyl, heteroalkenyl or heteroalkynyl chains, the chain being derivatized in two places with (identical or different) reactive groups. These groups form a covalent chemical bond in simple/known chemical reactions with the appropriate reaction partner. The reactive groups may also be photoactivatable, i.e. the reactive groups are activated only by light of a specific or any given wavelength. Preferred linkers are those having a chain length of 1-20, especially a chain length of 1-14, the chain length here representing the shortest continuous link between the structures to be joined, in other words between the two molecules or between a surface atom, surface molecule or surface molecule group and another molecule.
spacer Equivalent to linker.
mica Muscovite lamina, a support material for the application of thin films.
Au—S—(CH₂)₂-ss-oligo Gold film on mica having a covalently applied monolayer comprising derivatized single-strand oligonucleotide. Here, the oligonucleotide's terminal phosphate group at the 3′-end is esterified with (HO—(CH₂)₂—S)₂ to form P—O—(CH₂)₂—S—S—(CH₂)₂—OH, the S—S bond being homolytically cleaved and producing one Au—S—R bond each.
Au—S—(CH₂)₂-ds-oligo Au—S—(CH₂)₂-ss-oligo-spacer hybridized with the oligonucleotide that is complementary to the ss-oligo.
K_{A} The association constant for the association of ligate and ligand or ligate and signal ligand.
E The electrode potential on the working electrode.
E_{Ox} The potential at maximum current of the oxidation of a reversible electrooxidation or electroreduction.
i current density (current per cm² electrode surface)
cyclic voltammetry Recording a current-voltage curve. Here, the potential of a stationary working electrode is changed linearly as a function of time, starting at a potential at which no electrooxidation or electroreduction occurs, up to a potential at which a species that is dissolved or adsorbed to the electrode is oxidized or reduced (i.e. a current flows). Following completion of the oxidation or reduction operation, which produces in the current-voltage curve an initially increasing current and, after reaching maximum, a gradually decreasing current, the direction of the potential scan is reversed. The behavior of the products of electrooxidation or electroreduction is then recorded in a reverse run.
amperometry Recording a current-time curve. Here, the potential of a working electrode is set, for example by a potential jump, to a potential at which the electrooxidation or electroreduction of a dissolved or adsorbed species occurs, and the flowing current is recorded as a function of time.
chronocoulometry Recording a charge-time curve. Here, the potential of a working electrode is set, for example by a potential jump, to a potential at which the electrooxidation or electroreduction of a dissolved or adsorbed species occurs, and the transferred charge is recorded as a function of time. Chronocoulometry can thus be understood as an integral of amperometry.

The present invention provides a method for detection of ligate-ligand association events, comprising the steps: providing a modified surface, the modification consisting in the attachment of at least one type of ligate; providing signal ligands; providing a sample having ligands; bringing a defined quantity of the signal ligands into contact with the modified surface and bringing the sample into contact with the modified surface; detecting the signal ligands; and comparing with reference values the values obtained from the detection of the signal ligands.

Bringing a defined quantity of the signal ligands into contact with the modified surface and bringing the sample into contact with the modified surface may, in principle, also take place simultaneously, but preferably the signal ligands and the sample are brought into contact separately.

If a defined quantity of the signal ligands is brought into contact with the modified surface and, separately, the sample is brought into contact with the modified surface take place, there are two alternatives, namely bringing a defined quantity of the signal ligands into contact with the modified surface and thereafter bringing the sample into contact with the modified surface on the one hand, and on the other hand, first bringing the sample into contact with the modified surface and thereafter bringing a defined quantity of the signal ligands into contact with the modified surface. The present invention comprises both alternatives.

If a defined quantity of the signal ligands is brought into contact with the modified surface before the sample is brought into contact with the modified surface, according to a preferred embodiment of the present invention, the reference values may be determined each time before the sample is brought into contact with the modified surface. For this purpose, after a defined quantity of the signal ligands is brought into contact with the modified surface, a detection of the signal ligands is carried out and only thereafter is the sample brought into contact with the modified surface. Thereafter, the signal ligands are detected a second time, and the values determined in the second detection are compared with the reference values determined in the first detection. According to a particularly preferred embodiment, after the first detection of the signal ligands, the modified surface is washed and, after the sample is brought into contact with the modified surface, the same defined quantity of signal ligands is once more brought into contact with the modified surface. Only thereafter is the second detection of the signal ligands carried out. According to a further, particularly preferred embodiment, after the first detection of the signal ligands, but before the washing of the modified surface, conditions are set or actions taken that lead to at least predominant dissociation of ligates and signal ligands. In this way, during the subsequent wash step, as large a portion of the signal ligands as possible is removed from the surface.

If the sample is brought into contact with the modified surface before a defined quantity of the signal ligands is brought into contact with the modified surface, according to a preferred embodiment of the present invention, the reference values may be determined following detection of the signal ligands. For this purpose, first, conditions are set or actions taken that lead to at least predominant dissociation of ligates and ligands, then the modified surface is washed, thereafter signal ligands are brought into contact with the modified surface, the same defined quantity of signal ligands being used as for the first addition, and finally, the signal ligands are detected and the reference values thus determined.

One of the above-mentioned conditions that leads to increased dissociation of ligates and signal ligands, or to increased dissociation of ligates and ligands, is the addition of chaotropic salts, advantageously in a concentration of at least 3 mol/l. If oligonucleotides are used as ligate and as signal ligand, or as ligate and ligand, a temperature increase above the melting temperature of the ligate—signal ligand—oligonucleotide or of the ligate—ligand—oligonucleotide is also possible, especially to at least 5° C. above the melting temperature, furthermore the application of a potential that lies above the electrostringent potential, especially an increase in the (negative) potential by at least 10 mV above the electrostringent potential, as well as treatment with NaOH. If antigen (or antibody) and antibody (or antigen) are used as ligate and as signal ligand, or as ligate and ligand, increased dissociation can be accomplished e.g. by adding urea, advantageously in a concentration of at least 3 mol/l, or by adding a guanidinium salt, advantageously in a concentration of at least 3 mol/l.

The melting temperature of the ligate—signal ligand—oligonucleotide or of the ligate—ligand—oligonucleotide may either be determined under the appropriate external conditions, in other words with the given concentration of oligonucleotides, a specific salt content, a specific salt, in the presence of dissociation-promoting organic solvents such as formamide, DMF, etc., or calculated relatively precisely in broad ranges (see e.g. the oligo analyzer available on the Internet, www.idtdna.com/program/main/home.asp).

For electrode-immobilized ligate-oligonucleotides having an associated counter strand, the electrode potential may be used to displace the polyanionic counter strand from the electrode. As described for the melting temperature, this so-called electrostringent potential, too, may be determined experimentally for certain external parameters.

Examples of anions of chaotropic salts for dissociating double-strand oligonucleotides include CCl₃COO⁻, CNS⁻, CF₃COO⁻, ClO₄⁻, I⁻, (see also Robinson and Grant, Journal of Biological Chemistry, 241, 1966, p. 1329ff and Kessler et al., U.S. Pat. No. 5,753,433). Chaotropic salts lower the melting temperature.

According to the particularly preferred embodiments of the present invention described below, the separate execution of a reference measurement can be avoided. In these cases, on the modified surface are applied reference sites to which, after the sample is added, a very specific level of association can be assigned. The signal obtained from the detection is then characteristic for this specific level of association and can be consulted to standardize the signals of the test sites.

The present invention namely also comprises methods in which a modified surface is used that was modified by attaching at least two types of ligates. The differing types of ligates are bound to the surface in spatially substantially separate regions. The term “substantially separate regions” is understood to mean regions on the surface that are quite predominantly modified by attaching a specific type of ligate. Merely in areas in which two such substantially separate regions adjoin can it happen that differing types of ligate commingle. In the method preferred in the context of the present invention, before the sample is brought into contact with the modified surface, a ligand is added to the sample, the ligand being a binding partner having a high association constant to a specific type of ligate that is present bound to the surface in a specific region (test site T₁₀₀). Here, the ligand is added to the sample in a quantity that is greater than the quantity of ligands needed to completely associate the ligates of the T₁₀₀ test sites. The last step of this method is comparing with the value obtained for the T₁₀₀ region the values obtained from the detection of the signal ligands. The value obtained for the T₁₀₀ region thus corresponds to the value for complete association (100%).

According to a particularly preferred embodiment, a modified surface is used that was modified by attaching at least three types of ligates. The differing types of ligates are bound to the surface in spatially substantially separate regions. Here, in a specific region (test site T₀) is bound to the surface at least one type of ligate, about which it is known that no binding partner having a high association constant is contained in the sample, that is, the appropriate association partner or ligand does not occur in the sample. In this particularly preferred method, too, before the sample is brought into contact with the modified surface, a ligand is added to the sample, the ligand being a binding partner having a high association constant to a specific type of ligate that is present bound to the surface in a specific region (test site T₁₀₀). Here, the ligand is added to the sample in a quantity that is greater than the quantity of ligands needed to completely associate the ligates of the T₁₀₀ test sites. The last step of this method is comparing the values obtained from the detection of the signal ligands with the value obtained for the T₁₀₀ region and with the value obtained for the T₀ region. The value obtained for the T₀ region thus corresponds to the value for no association (0%).

According to a most particularly preferred embodiment of the two above-described methods, which do without a separate reference measurement, at least one additional type of ligand is added to the sample before the sample is brought into contact with the modified surface, it being known that this ligand is not contained in the original sample. This additional type of ligand exhibits an association constant >0 to one type of ligate, that is bound to the surface in a specific region (test site Tₙ). The ligand is added to the sample in such a quantity that, after the sample is brought into contact with the modified surface, n% of the ligates of the Tₙ test site are present in associated form. The last step of this method is comparing the values obtained from the detection of the signal ligands with the value obtained for the T₁₀₀ region, with the value obtained for the T₀ region and with the values obtained for the Tₙ regions. The value obtained for a specific test site Tₙ thus corresponds to the value for the presence of n% ligate-ligand associates based on the total number of ligates of the appropriate type.

The quantity of ligand that must be brought into contact with the modified surface to effect an n% association on the test site Tₙ can be determined by those of ordinary skill in the art through simple routine analyses. For this purpose, e.g. after detecting the values for T₀ and T₁₀₀, a calibrated measurement is carried out, in which is determined the signal intensity of (different) detection labels with which the ligate and the ligand are equipped. The ratio of ligand-label signal to ligate-label signal is n%.

If a sufficient number of Tₙ reference sites is applied on the modified surface, a reference curve can be recorded with great precision. Standardizing the measurements of the actual test sites with the aid of this reference curve significantly improves the reproducibility of chip-technology-aided analyses.

It should be pointed out that the discovery of a ligand that is not contained in the sample is no problem whatsoever, especially in the event that nucleic acid oligomers are used as ligands and ligates, since even the most extensive genomes still offer an adequate selection of sequences that are not present. In the event that the sequence that is not present differs from a sequence that is present by only one base, the hybridization step must be carried out under strict conditions. Preferably, however, sequences are used that differ significantly, i.e. in multiple bases, from the sequences that are present in the sample. Particularly good results are achieved when oligonucleotides having an identical or at least a similar number of bases are used for the test sites and for the reference sites.

Furthermore, the present invention is directed to the use of substrates, cofactors, coenzymes, proteins, enzymes, antibodies, antigens, receptors, hormones and nucleic acid oligomers as ligates and/or signal ligands and/or ligands in a method for detection of ligate-ligand association events.

In addition, the present invention is also directed to a kit for carrying out a method for detection of ligate-ligand association events. The kit comprises a modified surface, the modification consisting in the attachment of at least one type of ligate; and an effective quantity of signal ligands. With this kit, a two-fold detection of the signal ligands can be carried out, i.e. a determination of the reference values and the second detection of the signal ligands.

According to a preferred embodiment, the reference values are already comprised by the kit, so that the signal ligands must be detected by the end consumer only once. The values obtained from this detection then need only be compared with the already existing reference values.

According to a preferred embodiment of the present invention, the kit comprises a modified surface that exhibits at least one T₀ region and at least one T₁₀₀ region. Embodiments according to which the modified surface additionally comprises at least one Tₙ region are particularly preferred.

According to a preferred embodiment, the present invention thus provides a method for detection of ligate-ligand association events, such as sequence-specific nucleic acid oligomer hybridization events or antigen-antibody associates, based on a displacement assay. In doing so, ligates such as DNA/RNA/PNA oligomer single-strands or antigens immobilized on surfaces serve as an association matrix (probe) for detection of specific targets (e.g. specific oligonucleotides, DNA fragments or specific antigens). First, the ligates of the association matrix are brought into contact with a solution of signal ligands, causing some of the signal ligands to be complexed to the surface-immobilized ligates and the remaining signal ligands to remain in the excess solution. The signal ligands are chosen such that the surface associates comprising ligate and signal ligand possess an association constant that is less than the association constant between ligate and ligand. In addition, the signal ligands either function themselves as the signal-generating substance for detection, or they are labeled with a detectable signal-generating substance. In a first (reference) detection, the surface-immobilized signal ligands are captured using a suitable surface-sensitive measuring method (e.g. total internal reflection fluorescence or electrochemical methods such as chronocoulometry) that permits discriminating between surface-immobilized signal ligands and signal ligands in the volume phase. Thereafter, by adding to the modified surface the solution containing ligand to be analyzed, a portion of the signal ligands is displaced by the ligates, causing the ligate/signal ligand reference signal originally present in the system to be modulated and allowing qualitative and quantitative statements about ligands in the test solution. The association matrix may consist of only one test site with a ligate, but it is preferred that the association matrix consist of a plurality of test sites.

Thus, in principle, the determination of an unknown substance (ligand) takes place by detecting a third link (signal ligand) that, like the unknown substance (ligand), associates to a probe molecule (ligate). If signal ligand and ligand are present, due to the stronger association ability of the unknown substance (ligand), at least a portion of the signal ligands is displaced from the previously formed associate complex comprising signal ligand and probe molecule (ligate). Likewise, of course, if associates comprising ligand and ligate are present, a portion of the ligands may be displaced from the ligand-ligate complex by adding signal ligands. This occurs to a small degree despite the stronger association ability of ligand with ligate since, in any case, a balance will be reached that is determined by the ratio of the association constants of ligand/ligate to signal ligand/ligate.

The displacement assay thus comprises a complexation event between a ligate and a signal ligand, which is joined by a further complexation event after the actual target (ligand) is added, which takes place and displaces the signal ligand. Furthermore, the displacement assay comprises a complexation event between a ligate and a target (ligand), which is joined by a further complexation event after signal ligand is added, which may take place and displace at least a portion of the ligands.

The Surface

The term “surface” refers to any support material that is suitable for binding derivatized or non-derivatized ligates covalently or via other specific interactions, directly or following appropriate chemical modification. The solid support may consist of conductive or non-conductive material.

(i) Conductive Surfaces

The term “conductive surface” is understood to mean any support having an electrically conductive surface of any thickness, especially surfaces comprising platinum, palladium, gold, cadmium, mercury, nickel, zinc, carbon, silver, copper, iron, lead, aluminum and manganese.

In addition, any doped or undoped semiconductor surfaces of any thickness may also be used. All semiconductors may be used in the form of pure substances or in the form of composites. Examples include, but are not limited to, carbon, silicon, germanium, α tin, and Cu(I) and Ag(I) halides of any crystal structure. All binary compounds of any composition and any structure comprising the elements of groups 14 and 16, the elements of groups 13 and 15, and the elements of groups 15 and 16 are also suitable. In addition, ternary compounds of any composition and any structure comprising the elements of groups 11, 13 and 16 or the elements of groups 12, 13 and 16 may be used. The designations of the groups of the periodic table of the elements refer to the IUPAC recommendation of 1985.

(ii) Non-Conductive Surfaces

The material preferred for non-conductive surfaces is glass and modified glass. The modification may take place e.g. by silanization and, in all cases, results in functional groups that are suitable for binding, in coupling reactions, appropriately functionalized ligate molecules. This modification includes layered superstructures on the glass surface when using polymers, such as dextran polymers, that allow a variation of the layer thickness and surface condition. Further derivatization options of the glass for the ultimate attachment of the ligates consist, e.g., in applying a thin (approximately 10-200 nm) metallization layer, especially a gold metallization layer, which may additionally be coated with (thiol-functionalized) polymers, especially dextrans. In addition, following silanization, the glass may also be functionalized with biotin (e.g. amino-functionalized glass surface following silanization and coupling of the carboxylic acid biotin via EDC and NHS or via a biotin active ester such as biotin-N-succinimidyl ester) or, alternatively, coated with a dextran lysine or dextran-immobilized biotin. Thereafter, the biotinylated glass surfaces produced in this way are treated with avidin or streptavidin and may then be used for the attachment of biotinylated ligate molecules.

Binding the Ligate to the Surface

Methods for immobilization of ligate molecules, especially biopolymers such as nucleic acid oligomers, antigens, antigen-antibody complexes or antibodies, to a surface are known to those of ordinary skill in the art. The ligate molecules may, e.g., be covalently bound to the surface via hydroxyl, epoxide, amino or carboxy groups of the support material having thiol, hydroxy, amino or carboxyl groups that are naturally present on the ligate or that have been affixed to the ligate by derivatization. The ligate may be bound to the surface atoms or molecules of a surface directly or via a linker/spacer. In addition, the ligate may be anchored by the methods common in immunoassays, such as by using biotinylated ligates for non-covalent immobilization to avidin or streptavidin-modified surfaces. If nucleic acid oligomers are used as ligates, the chemical modification of the ligate nucleic acid oligomers with a surface anchor group may be introduced as early as in the course of automated solid-phase synthesis, or in separate reaction steps. In this process, the nucleic acid oligomer is also linked directly or via a linker/spacer with the surface atoms or surface molecules of a surface of the type described above. This bond may be accomplished in various ways (cf. e.g. WO 00/42217).

Ligands/Signal Ligands

Molecules that specifically interact with the ligate that is immobilized to a surface, forming a complex, are referred to as ligands. Examples of ligands within the meaning of the present invention are substrates, cofactors or coenzymes, as complex binding partners of a protein (enzyme), antibodies (as complex binding partners of an antigen), antigens or antigen-antibody complexes (as complex binding partners of an antibody), receptors (as complex binding partners of a hormone), hormones (as complex binding partners of a receptor) and nucleic acid oligomers (as complex binding partners of the complementary nucleic acid oligomer).

In the context of the present invention, molecules that, themselves or following appropriate modification with a detection label, can be determined by suitable detection methods are referred to as signal ligands. Detection methods that may be used are the measurement of fluorescence or electrochemiluminescence, or electrochemical detection. In the context of the present invention, the signal ligands possess a lower tendency to form complexes with the ligates than do the actual ligands (targets), i.e. the association constant between ligand and ligate is greater than the association constant between signal ligand and ligate.

Examples of signal ligands are
detection-labeled antibodies (as signal complex binding partners of an antigen) whose association constant with the ligate antigen is lower than the association constant of ligate antigen and ligand antibody,
detection-labeled antigen (as the signal complex binding partner of an antibody) whose association constant with the ligate antibody is lower than the association constant of ligate antibody and ligand antigen,
detection-labeled receptors (as signal complex binding partners of a hormone) whose association constant with the ligate hormone is lower than the association constant of ligate hormone and ligand receptor,
detection-labeled hormone (as the signal complex binding partner of a receptor) whose association constant with the ligate receptor is lower than the association constant of ligate receptor and ligand hormone,
detection-labeled nucleic acid oligomers (as signal complex binding partners of the complementary nucleic acid oligomer) whose association constant with the ligate nucleic acid oligomer (probe nucleic acid oligomer) is lower than the association constant of ligate nucleic acid oligomer and ligand nucleic acid oligomer. A detection-labeled nucleic acid oligomer with fewer than n complementary nucleic acids may be used, e.g., for a ligate oligonucleotide with n nucleotides. Thus, in the case of a ligate oligonucleotide with 20 bases, the signal oligonucleotide exhibits only nucleic acid regions in which fewer than 20 consecutive bases of the signal oligonucleotide are complementary to the 20 bases of the ligate oligonucleotide. Thus, for a ligate oligonucleotide with 20 nucleotides, a detection-labeled signal oligonucleotide of any length may be used, as long as the signal oligonucleotide exhibits only nucleic acid regions in which fewer than 20 consecutive bases of the signal oligonucleotide are complementary to the 20 bases of the ligate oligonucleotide. In particular, the signal oligonucleotide may be a 20-nt oligo labeled with at least one detection label, or contain a 20-nt oligo sequence that is complementary to the 20-nt ligate oligonucleotide and that forms one or more base-pair mismatches during complexation between signal oligonucleotide and ligate oligonucleotide. An oligonucleotide that exhibits fewer than 20 nt and that is completely complementary to the ligate oligonucleotide and labeled with at least one detection label may also be used,
detection-labeled single-stranded DNA binding protein (as the signal complex binding partner of a nucleic acid oligomer) whose association constant with the ligate nucleic acid oligomer is lower than the association constant of ligate nucleic acid oligomer and ligand nucleic acid oligomer, e.g. p10 single-stranded nucleic acid binding protein with K_{A}=10⁸ (W. J. Roberts et al., Synthesis of the p10 single-stranded nucleic acid binding protein from murine leukemia virus, Pept. Res. 1 (1988) 74-80) and a nucleic acid oligomer of length 20 nt with K_{A}=10²⁰,
detection-labeled nucleic acid oligomers (as signal complex binding partners of an antibody) whose association constant with the ligate antibody is lower than the association constant of ligate antibody and ligand nucleic acid oligomer,
detection-labeled nucleic acid oligomers (as signal complex binding partners of an antigen) whose association constant with the ligate antigen is lower than the association constant of ligate antigen and ligand nucleic acid oligomer,
detection-labeled antibodies (as signal complex binding partners of a nucleic acid oligomer) whose association constant with the ligate nucleic acid oligomer is lower than the association constant of ligate nucleic acid oligomer and ligand antibody,
detection-labeled antigen (as the signal complex binding partner of a nucleic acid oligomer) whose association constant with the ligate nucleic acid oligomer is lower than the association constant of ligate nucleic acid oligomer and ligand antigen.

In the context of the present invention, a compound comprising at least two covalently-joined nucleotides or at least two covalently-joined pyrimidine (e.g. cytosine, thymine or uracil) or purine bases (e.g. adenine or guanine), preferably a DNA, RNA or PNA fragment, is used as the nucleic acid oligomer. The term nucleic acid refers to any backbone of the covalently-joined pyrimidine or purine bases, such as the sugar-phosphate backbone of DNA, cDNA or RNA, a peptide backbone of PNA, or analogous backbone structures, such as a thiophosphate, a dithiophosphate or a phosphoramide backbone. An essential feature of a nucleic acid within the meaning of the present invention is the sequence-specific binding of naturally occurring cDNA or RNA.

Detection Label/Marker (Marker Molecule)

Signal ligands that cannot be detected themselves are provided with a detectable label by derivatization. This label allows detection of the complexation events between the signal ligand and the surface-bound ligate. The label can supply a detection signal directly or, as in the case of enzyme-catalyzed reactions, indirectly. Preferred detection labels (marker molecules) are fluorophores and redox-active substances.

For the fluorophores, commercially available fluorescent dyes such as Texas Red, rhodamine dyes, fluorescein, etc. are used (cf. Molecular Probes Catalog). In the event of detection by electrochemical methods, redox molecules are employed as labels. Transition metal complexes, especially those of copper, iron, ruthenium, osmium or titan may be used as redox labels with ligands such as pyridine, 4,7-dimethylphenanthroline, 9,10-phenanthrene quinonediimine, porphyrins and substituted porphyrin derivatives. In addition, it is possible to employ riboflavin, quinones such as pyrrolloquinoline quinone, ubiquinone, anthraquinone, naphthoquinone or menaquinone, or derivatives thereof, metallocenes and metallocene derivatives such as ferrocenes and ferrocene derivatives, cobaltocenes and cobaltocene derivatives, porphyrins, methylene blue, daunomycin, dopamine derivatives, hydroquinone derivatives (para- or ortho-dihydroxybenzene derivatives, para- or ortho-dihydroxyanthraquinone derivatives, para- or ortho-dihydroxynaphthoquinone derivatives) and similar compounds.

Surface-Sensitive Detection Methods

Surface-sensitive detection methods allow discrimination between marker molecules associated to a surface and those dissolved in excess. Electrochemical, spectroscopic and electrochemiluminescent methods are suitable as the detection method.

(i) Surface-Sensitive Electrochemical Detection

In electrochemical methods, in principle, the kinetics of the electrochemical processes may be used to discriminate between redox-active detection labels adsorbed to a surface and those dissolved in excess. Generally, surface-adsorbed detection labels are electrochemically converted (e.g. oxidized or reduced) more quickly than redox-active detection labels from the volume phase, since the latter must first diffuse to the (electrode) surface before electrochemical conversion. Examples of electrochemical surface-sensitive methods include amperometry and chronocoulometry.

The chronocoulometry method allows discriminating surface-near redox-active components from (identical) redox-active components in the volume phase and is described, for example, in Steel, A. B., Herne, T. M. and Tarlov M. J.: Electrochemical Quantitation of DNA Immobilized on Gold, Analytical Chemistry, 1998, Vol. 70, 4670-4677 and the literature cited therein.

The chronocoulometry measurement signal (transferred charge Q as a function of time t) is composed of three components: (i) a diffusive portion, which is brought about by the dissolved redox-active components in the volume phase and exhibits a t^{1/2} dependence, a first instantaneous portion, which results from the charge redistribution in the double layer (dl) on the electrode surface, and a second instantaneous portion, which is caused by the conversion of redox-active components adsorbed (immobilized) on the electrode surface.

In the chronocoulometric experiment, the Cottrell equation gives the charge Q as a function of time t:

$$Q = \frac{2 {nFAD}_{0}^{1 / 2} {C}_{0}^{*}}{{π}^{1 / 2}} {t}^{1 / 2} + {Q}_{dl} + n FA {Γ}_{0}$$

wherein
n: number of electrons per molecule for the reduction
F: Faraday constant
A: electrode surface [cm²]
D₀: diffusion coefficient [cm²/s]
C₀^{*}: concentration [mol/cm²]
Q_{dl}: capacitive charge C
nFAΓ₀: charges that are converted during the electrochemical conversion of the adsorbed redox-active detection label, wherein Γ₀ [mol/cm²] represents the surface concentration of the detection label.

The term Γ₀ thus represents the quantity of detection label on the electrode surface. In the chronocoulometric experiment, when t=0, the sum of the double-layer charges and the surface excess is maintained.

A chronocoulometrically detected displacement assay within the meaning of the present invention will be explained using the example of a 20-nt probe nucleic add oligomer as the ligate. The (working) electrode modified with 20-nt ligate oligonucleotides is brought into contact with a defined quantity of signal ligands, e.g. a 12-nt signal nucleic acid oligomer that carries one or more redox labels and is complementary to a region of the ligate oligonucleotide that is as close to the surface as possible so that association can occur between ligate oligonucleotide and redox-labeled ss nucleic acid oligomer complex former. Thereafter, the (working) electrode is initially set to a potential E₁ at which little to no electrolysis (electrochemical change in the redox state) of the redox label can occur (e.g., for ferrocene-modified ligate oligonucleotide, approximately 0.1 V versus Ag/AgCl (sat. KCl)). Then the working electrode is set by a potential jump to a potential E₂ at which the electrolysis of the redox label occurs in the diffusion-limited borderline case (e.g., for ferrocene-modified ss nucleic acid oligomer complex former, approximately 0.5 V versus Ag/AgCl (sat. KCl)). The transferred charges are recorded as a function of time.

Thereafter, the sample solution is added that is supposed to (may) contain the ligand oligonucleotide that exhibits an nt sequence that, in one region, is complementary to the 20 nt of the ligate oligonucleotides. Following hybridization of the target to the ligate oligonucleotides, and thus following partial displacement of the signal ligands, a second electrochemical measurement is carried out. The change in the instantaneous charge signal is proportional to the number of displaced signal oligonucleotides and is thus proportional to the number of target oligonucleotides present in the test solution.

This change in the instantaneous charge signal depends on the length of the ligand oligonucleotides, that is, on the number of bases of the ligand oligonucleotides. If the ligand oligonucleotides exhibit a length that approximately corresponds to or is shorter than the length of the ligate oligonucleotides, a decrease in the instantaneous charge signal will be observed, since signal oligonucleotides are displaced by the ligand oligonucleotides from the associate with the ligate oligonucleotides, and thus from the proximity of the modified surface. If the ligand oligonucleotides exhibit a longer length than the ligate oligonucleotides, only a portion of the bases of the ligand oligonucleotides can attach to the bases of the ligate oligonucleotides, and an excess portion of the ligand oligonucleotide with freely accessible bases remains. Generally, following hybridization of the ligand oligonucleotides to the ligate oligonucleotides, the signal oligonucleotides attach to these free bases of the ligand oligonucleotides. If the excess portion of the ligand oligonucleotides is located near the surface, it may happen that an increase in the instantaneous charge signal is observed, since the number of signal oligonucleotides located near the surface increases through the addition of the long-chain ligand oligonucleotides and binding of the signal oligonucleotides to these ligand oligonucleotides. For very long-chain ligand oligonucleotides, a considerably greater difference (increase) may result between the instantaneous charge signal before the addition of ligand oligonucleotide and the instantaneous charge signal after the addition of ligand oligonucleotide than for short-chain ligand oligonucleotides (decrease in the instantaneous charge signal). Uncertain results are obtained only when the number of signal ligands displaced from the signal ligand oligonucleotide/ligate oligonucleotide associate approximately corresponds to the number of signal ligands that subsequently attach to the portion of the ligand oligonucleotides that are located near the surface and that exceeds the portion associated to the ligate oligonucleotide.

(ii) Surface-Sensitive Fluorescence Detection

Total internal reflection fluorescence (TIRF, cf. Sutherland and Dahne, 1987, J. Immunol. Meth., 74, 253-265) can serve as an optical measuring method for detection of fluorescence-labeled signal nucleic acid oligomer ligands. Here, fluorescence molecules that are located near the interface between a solid waveguide medium, typically glass, and a liquid medium, or that are immobilized on the waveguide medium surface facing the liquid, can be excited by the evanescent field protruding from the waveguide and emit detectable fluorescence light. Fluorescence-labeled complex formers that are displaced or dissolved in the excess are not captured by the evanescent field (or only to the extent that they are located in the range of the penetration depth of the evanescent field) and thus contribute (nearly) nothing to the measured signal. The penetration depth of the evanescent field is typically 100 to 200 nm, but it may also be increased by a thin metallization layer (approximately 10-200 nm), especially a gold metallization layer, to several 100 nm. In a preferred embodiment of the fluorescence detection of the displaced, fluorophore-labeled signal ligands, the layer thickness of the ligate-modified support surface is adjusted to the penetration depth of the evanescent field, e.g.
by appropriately large ligates, especially appropriately long ligate oligonucleotides,
by immobilizing the ligates via appropriately long linkers between surface and ligate, especially appropriately long linkers between ligate oligonucleotide and surface,
by coupling the carboxylic acid biotin (via EDC and NHS or via a biotin active ester such as biotin-N-succinimidyl ester) to amino-derivatized surfaces and coupling avidin or streptavidin to the biotinylated surfaces produced in this way, with subsequent attachment of biotinylated ligate molecules or
by immobilizing an appropriately thick layer on functionalized polymer and attaching the ligate to the polymer, e.g. (a) by applying a thin (approximately 10-200 nm) metallization layer, especially a gold metallization layer, which may be coated with a (thiol-functionalized) polymer, especially dextrans or polylysine, which, in turn, is used for the attachment of the ligate, or (b) by applying a polymer layer comprising polylysine-biotin, dextran-lysine biotin or dextran-immobilized biotin and coupling avidin or streptavidin to the biotinylated surfaces produced in this way, with subsequent attachment of biotinylated ligate molecules.

### BRIEF DESCRIPTION OF THE DRAWINGS

The invention will be explained in greater detail below by reference to exemplary embodiments in association with the drawings, wherein:

FIG. 1 Shows a schematic diagram of the detection of complex-formation events by means of a displacement assay;

FIG. 2 Shows a cyclovoltammogram of ferrocene carboxylic acid (gold working electrode, platinum counter electrode, Ag/AgCl (sat. KCl) reference electrode, 10 mM ferrocene carboxylic acid);

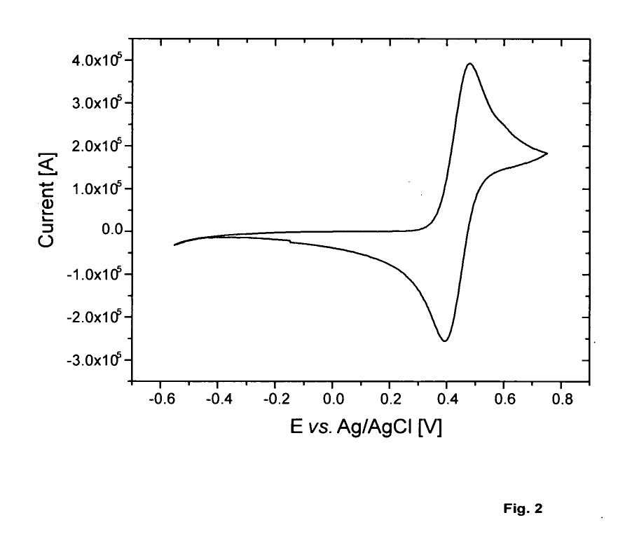

FIG. 3 Shows a chronocoulometric measurement of the sequence-specific hybridization of a 20-mer nucleic acid ligate with the complementary counter strand (ligand) by detecting the ferrocene-labeled tetramer signal ligands displaced by hybridization.

### REFERENCE SIGNS

A: Array having ligate molecules immobilized via a suitable linker x (two test sites)
B: Array incubated with a solution of signal ligands
C: Array having signal ligands associated to ligate molecules
D: Array having specific complexation of a test site with target
E: Array having specific complexation of a ligate with target and with signal ligands associated to a ligate
101: Ligate 1
102: Ligate 2
103: Signal ligand, e.g. oligonucleotide
104: Detection label, e.g. ferrocene
105: Surface, e.g. gold
106: Ligand
Step 1: Addition of a signal ligand
Step 2: Complex formation, e.g. hybridization
Step 3: Dissociation and removal of the signal ligands and subsequent addition of the ligand
Step 4: Addition of the ligand
Step 5: Addition of the signal ligands

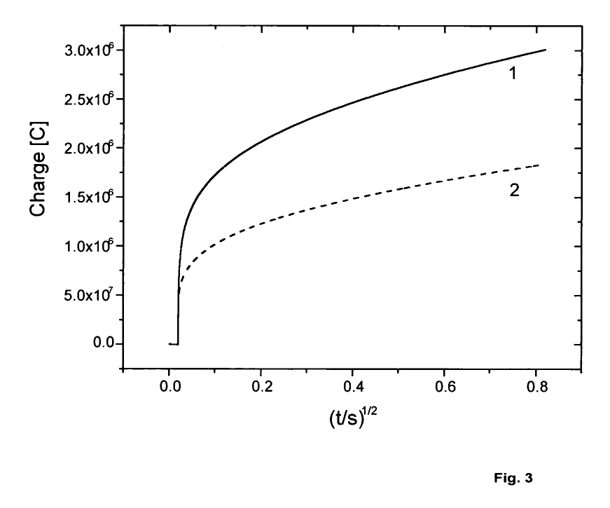

In FIG. 3, the curve labeled “1” displays the chronocoulometric measurement following hybridization of the ferrocene-labeled tetramer signal ligands, while the curve labeled “2” shows the chronocoulometric measurement following addition of the complementary ligand and (partial) displacement of the redox-labeled tetramers from the ligate surface.

### MANNER OF EXECUTING THE INVENTION

Alternative Methods of the Displacement Assay for Detection of Ligate/Ligand Associates

Alternative methods of the displacement assay will be highlighted below using the example of detection of nucleic acid oligomer hybridization events. For those of ordinary skill in the art, the alternative methods can easily be carried over to the detection of other ligate/ligand associates, such as those mentioned in the section “Ligands/Signal Ligands.”

To apply the advantages of DNA chip technology to the detection of nucleic acid oligomer hybrids by the displacement assay, various modified ligate nucleic acid oligomers of differing in sequence are bound to a support with the above-described immobilization techniques. The arrangement of the ligate nucleic acid oligomers of known sequence at defined positions on the surface, a DNA array, is intended to make the hybridization event of any ligand nucleic acid oligomer or of a (fragmented) ligand DNA detectable in order to e.g. seek and sequence-specifically detect mutations in the ligand. For this purpose, the surface atoms or molecules of a defined region (a test site) on a surface are linked with DNA/RNA/PNA nucleic acid oligomers of a known but arbitrary sequence as described above. The DNA chip may also be derivatized with a single ligate oligonucleotide. Preferred ligate nucleic acid oligomers are nucleic acid oligomers (e.g. DNA, RNA or PNA fragments) of base length 3 to 70 or 3 to 50, preferably of length 8 to 50 or 5 to 30, particularly preferably of length 10 to 30 or 8 to 25.

The surface thus provided having immobilized ligate oligonucleotides is incubated with a solution of a specific quantity of signal ligands, e.g. redox-labeled nucleic acid oligomers, that are hybridizable only to a specific sequence section of the ligate oligonucleotide, but not to the entire sequence of the ligate oligonucleotide. This leads to the formation of hybrids comprising ligate nucleic acid and the signal ligand oligonucleotides in the region of complementary sequences.

Following hybridization between ligate and signal ligand, in a reference measurement, the surface-immobilized portion of the signal ligands is determined (e.g. by a first chronocoulometric measurement, cf. the section “Surface-sensitive Detection Methods”).

In the next step, the test solution (as concentrated as possible) with ligand oligonucleotide(s) is added to the surface having immobilized ligate oligonucleotides, associated signal ligands and excess solution (with free, non-surface-adsorbed signal ligands). This leads to hybridization only if the solution contains ligand nucleic acid oligomer strands that are complementary to the ligate nucleic acid oligomers bound to the surface, or complementary in at least in wide regions (or wider regions than the signal oligonucleotide). The originally associated signal oligonucleotides are, at least partially, displaced.

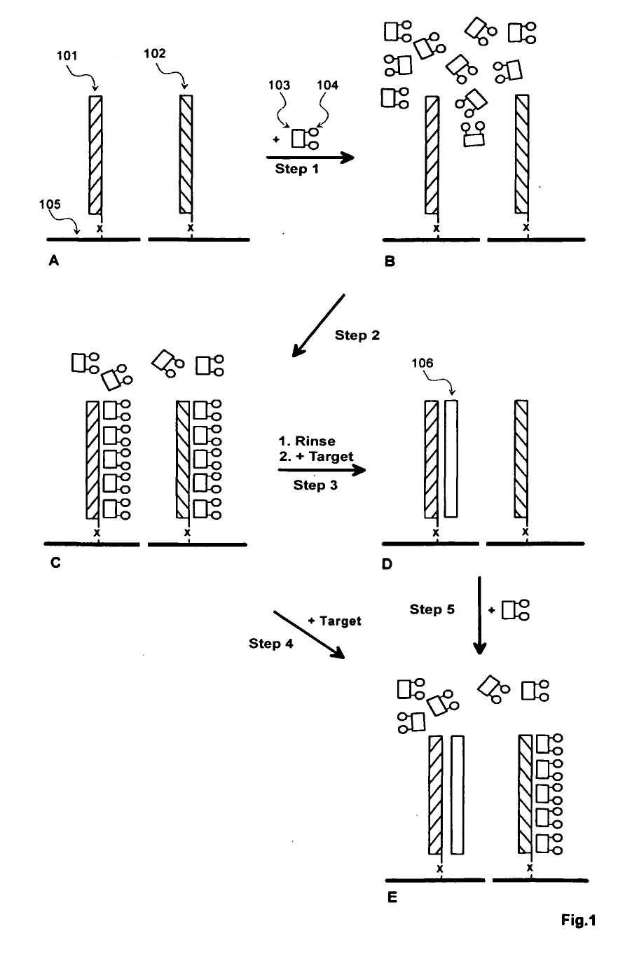

Following hybridization between ligate and ligand, in a second measurement (e.g. a second chronocoulometric measurement, cf. the section “Surface-sensitive Detection Methods”), the portion of remaining surface-immobilized signal ligands is determined. The difference between reference measurement and second measurement for each test site is proportional to the number of complementary (or complementary in wide regions) ligand oligonucleotides originally present in the test solution for the relevant test site (cf. FIG. 1, procedure with steps 1, 2, 4).

Alternatively, following the reference measurement, the associates comprising ligate and signal ligand on the surface may be dissociated, e.g. by increasing the temperature, and all signal ligands or only the signal ligands in the excess solution may be removed by washing such that, following the reference measurement, the originally employed surface having immobilized ligate oligonucleotides is available. The removal of the signal ligands from the associates with the ligates generally takes place by taking the modified surface out of the solution containing the signal ligands and subsequently washing the modified surface. In doing so, dehybridizing conditions may be set when washing the modified surface and/or before removing the modified surface from the solution containing the signal ligands. In the next step, the sample solution with ligand oligonucleotide(s) is added to the surface having immobilized ligate oligonucleotides and the potentially present ligand oligonucleotides may be hybridized to the ligate oligonucleotides under any stringency conditions known to those of ordinary skill in the art. Ideally, by setting the stringency conditions, it can be achieved that exclusively complementary ligand oligonucleotides remain hybridized to the ligate oligonucleotides, whereas “ligand oligonucleotides” that exhibit one or more mismatches dehybridize. Excess test solution with unattached ligands is removed by washing with suitable buffer solutions. Thereafter—as for carrying out the reference measurement—incubation with the solution containing a specific quantity of signal ligands again takes place, and in the second measurement, the portion of the signal ligands still associating to the ligate oligonucleotides is determined (cf. FIG. 1, procedure with steps 1, 2, 3, 5).

In a further alternative, the reference measurement may be omitted if the size of the reference signal is known sufficiently precisely beforehand (e.g. through preceding measurements, etc.). Here, first the hybridization with ligands and, thereafter, step 5 is carried out (cf. FIG. 1, procedure with step 5). In this case, first the sample solution with ligand oligonucleotides is added to the surface having immobilized ligate oligonucleotides and hybridized, if necessary under strict conditions. Thereafter, a solution that contains a specific quantity of signal ligands is added. Then, with the aid of a measurement, the portion of signal ligands that is present associated to the ligate oligonucleotides is determined, and the values obtained are compared with the known reference signal.

### EMBODIMENTS

(i) Covalent Embodiment with Attachment of Ligate Oligonucleotides to (One) Individually Addressable Gold Electrode(s), Redox-Labeled na Tetramers as Signal Ligands, Ligand Oligonucleotides and Chronocoulometric Detection of the Displacement of the Signal Ligands by the Ligands:

The n-nucleotide (nt)-long ligate nucleic acid (DNA, RNA or PNA, e.g. a 20-nucleotide-long oligo) (FIG. 1A, 101 or 102) is provided near one of its ends (3′- or 5′-end), directly or via a (any) spacer, with a reactive group for covalent anchoring to the surface, e.g. 3′-thiol-modified ligate oligonucleotide in which the terminal thiol modification serves as a reactive group for attachment to gold electrodes. Further covalent anchoring options result from e.g. amino-modified ligate oligonucleotide, which is used for anchoring to glass carbon electrodes that are superficially oxidized onto carboxylic acid, or to platinum electrodes. In addition, a monofunctional linker of suitable chain length with an identical reactive group may be provided. The ligate nucleic acid modified in this way is,
(i) dissolved in buffer (e.g. 50-500 mM phosphate buffer, pH=7, 1 mM EDTA), brought into contact with the surface and attached there via the reactive group of the ligate nucleic acid oligomer to the—if necessary, appropriately derivatized—surface or
(ii) dissolved in the presence of a monofunctional linker in buffer (e.g. 100 mM phosphate buffer, pH=7, 1 mM EDTA, 0.1-1 M NaCl), brought into contact with the surface and attached there via the reactive group of the ligate nucleic acid oligomer, together with the monofunctional linker, to the—if necessary, appropriately derivatized—surface, care being taken that sufficient monofunctional linker of suitable chain length is added (about 0.1- to 10-fold or even 100-fold excess) to provide between the individual ligate oligonucleotides sufficient space for hybridization with the redox-labeled signal ligands or the ligand oligonucleotide, or
(iii) dissolved in buffer (e.g. 10-350 mM phosphate buffer, pH=7, 1 mM EDTA), brought into contact with the surface and attached there via the reactive group of the ligate nucleic acid oligomer to the—if necessary, appropriately derivatized—surface. Thereafter, the surface modified in this way is brought into contact with the appropriate monofunctional linker in solution (e.g. alkanethiols or ₁₃ -hydroxy-alkanethiols in phosphate buffer/EtOH mixtures for thiol-modified ligate oligonucleotides), the monofunctional linker attaching via its reactive group to the—if necessary, appropriately derivatized—surface (cf. the section “The Surface”).

The surface modified in this way (FIG. 1A) is brought into contact with redox-labeled nucleic acid oligomers (FIG. 1B, 104) comprising fewer than n complementary nucleotides (FIG. 1B, 103). As redox-labeled signal ligands, e.g. singly or multiply ferrocene-carboxylic acid-modified nucleic acid tetramers (cf. ex. 3) whose sequence is complementary to tetramer partial sequences of the ligate oligonucleotides may be used, or SSB (single-stranded DNA-binding protein) modified with redox label (e.g. ferrocene derivatives) may be employed. Here, care is taken that significantly more labeled signal ligands (at least 1.1-fold molar excess) are added than can be bound to the surface via the ligate nucleic acid oligomers.

The detection label on the signal ligand is detected by a suitable method, e.g. chronocoulometry in the case of the ferrocene-redox-labeled signal oligonucleotides. Thereafter, the dissolved ligand is added and the measurement for detecting the detection label is repeated with the suitable method (e.g. renewed chronocoulometric measurement in the case of the ferrocene-redox-labeled signal oligonucleotides).

Alternatively, the modified surface is removed from the solution containing signal ligands, if necessary washed as described above and subsequently brought into contact with the solution containing the ligands. The hybridization may be carried out under suitable conditions known to those of ordinary skill in the art (any, freely selectable stringency conditions for the parameters potential/temperature/salt/chaotropic salts, etc., for hybridization). Thereafter, the modified surface is brought into contact with signal ligands (in the same concentration as for the preceding measurement) and a measurement for detecting the detection label is carried out with a suitable method.

The difference in the measurement signal (decrease or increase, depending on the measuring method) is proportional to the number of hybridization events between ligate nucleic acid oligomer on the surface and matching ligand nucleic acid oligomer in the test solution (cf. ex. 6). In chronocoulometric measurement, a decrease is detected in the nearly instantaneous signal portion of the surface excess of redox labels, cf. “Surface-sensitive Detection Methods.”

As a variant of the described method, after the first detection (reference measurement) has taken place, the complexes comprising surface-bound ligate and labeled signal ligands (FIG. 1C) are dissolved (e.g. by temperature increase) and the free, labeled signal ligands (oligonucleotides) are removed from the excess by washing (FIG. 1, Step 3). Following addition of the nucleic acid ligand, hybridization (FIG. 1D) and addition of the labeled nucleic acid oligomer signal ligands (FIG. 1, Step 5), a second chronocoulometric measurement (FIG. 1E) is carried out.

The method may be applied for one ligand type (e.g. a specific ligand oligonucleotide type having a known sequence) on one electrode; or for multiple ligand types (e.g. differing ligand oligonucleotide types or differing antibody types, antigen types, etc., but also mixtures thereof) on individually addressable electrodes of an electrode array that can be targeted and read out, e.g. via CMOS technology, in more complex arrays.

(ii) Embodiment with Indirect Attachment of Ligate Oligonucleotides to Glass Fibers, with Multiply Fluorophore-Labeled na Dodecamers as Signal Ligands, Ligand Oligonucleotides and Fluorescence Detection of the Displacement of the Signal Ligands by the Ligands:

The n-nucleotide-long ligate nucleic acid (DNA, RNA or PNA) (FIG. 1A, 101 or 102, e.g. a 20-nucleotide-long oligo), is provided near one of its ends (3′- or 5′-end), directly or via a (any) spacer, with a reactive group for covalent anchoring to the surface, e.g. a carboxy-modified ligate oligonucleotide for attachment to amino-modified silanized glass (e.g. to (3-aminopropyl)-triethoxysilane-modified glass). Further covalent anchoring options result from e.g. amino-modified ligate oligonucleotide, which is used for anchoring to dextran polymers that are derivatized with carboxylic acid and immobilized on glass, the thickness of the layer comprising dextran polymer with attached ligate oligonucleotides in this embodiment being able to be varied via the dextran polymer composition, dextran anchor groups on the glass surface, anchor groups of the dextran for immobilization on the glass, incubation duration on the glass, etc., using methods known to those of ordinary skill in the art. In a preferred embodiment, the thickness of the dextran/ligate oligonucleotide layer is chosen such that it approximately corresponds to the penetration depth of the evanescent field of the light for the excitation of the fluorophores (approx. 50 nm to approx. 500 nm, depending on whether a metallization layer is located on the glass to increase the penetration depth of the evanescent field). The ligate nucleic acid modified in this way is dissolved in buffer (e.g. 50-500 mM phosphate buffer, pH=7, 1 mM EDTA) in the presence of EDC and sNHS (each approximately 40-fold molar excess in reference to the ligate oligonucleotide), brought into contact with the modified glass surface and attached there to the surface via the reactive group of the ligate nucleic acid oligomer (if necessary, nonfunctional surface binding sites are treated beforehand with a suitable blocking reagent).

The surface modified in this way (FIG. 1A) is brought into contact with multiply fluorophore-labeled signal ligands (FIG. 1B, 104) comprising fewer than n complementary nucleotides (FIG. 1B, 103). As fluorophore-labeled signal ligands, e.g. fluorescein-modified nucleic acid dodecamers may be used whose sequences are complementary to dodecamer partial sequences of the ligate oligonucleotides, or SSB (single-stranded DNA-binding protein) modified with one or more fluoresceins (e.g. FITC derivatives) may be employed. Here, care is taken that significantly more labeled signal ligands (at least 1.1-fold molar excess) are added than can be bound to the surface via the ligate nucleic acid oligomers.

The detection label on the signal ligand oligonucleotide is detected by a suitable method, e.g. total internal reflection fluorescence (TIRF) in the case of the fluorophore-labeled signal oligonucleotides. Thereafter, the signal ligand in the excess solution is removed by washing, the test solution is added and potential hybridization events are facilitated under suitable conditions known to those of ordinary skill in the art (any, freely selectable stringency conditions for the parameters potential/temperature/salt/chaotropic salts, etc., for hybridization). After that, the quantity of signal ligand originally employed for the reference measurement is again added and the measurement for detecting the detection label is repeated with the suitable method (e.g. renewed TIRF measurement in the case of the fluorescein-labeled signal oligonucleotides). The difference in the measurement signal (decrease or increase, depending on the measuring method) is proportional to the number of hybridization events between ligate nucleic acid oligomer on the surface and matching ligand nucleic acid oligomer in the test solution (cf. ex. 6). For detection by determining the TIRF, a decrease in the fluorescence signal is to be expected.

As a variant of the described method, after the first detection (reference measurement) has taken place, the complexes comprising surface-bound ligate and labeled nucleic acid oligomers (FIG. 1C) are dissolved and the free, labeled oligonucleotides are removed from the excess by washing (FIG. 1, Step 3). Following addition of the nucleic acid ligands, hybridization (FIG. 1D) and addition of the labeled nucleic acid oligomers (FIG. 1, Step 5), a second measurement (FIG. 1E) is then carried out.

The method may be applied for one ligand type (e.g. a specific ligand oligonucleotide type having a known sequence) on a glass fiber; or for multiple ligand types (identical ligand groups such as differing ligand oligonucleotide types or differing antibody types, antigen types, etc., but also mixtures thereof) on individually addressable glass fibers of a glass fiber bundle.

### EXAMPLE 1

### Constituting the N-hydroxysuccinimide Active Ester of the Redox (or Fluorophore) Label

1 mmol of the relevant carboxylic acid derivative of a fluorophore (e.g. fluorescein) or of a redox-active substance (e.g. ferrocene) and 1.1 mmol N-hydroxysuccinimide are dissolved in 15 ml anhydrous dioxane. 1.1 mmol carbodiimide (dissolved in 3 ml anhydrous dioxane) are cooled with ice and added dropwise to the carboxylic acid derivative. The reaction mixture is stirred for 16 h at RT, the resultant precipitate filtered off and the solvent drawn off. The residue is purified by silica gel chromatography (Merck silica gel 60, solvent system: dichloromethane/ethyl acetate/heptane mixtures).

### EXAMPLE 2

### Constituting the Amino-Modified Oligonucleotides for Coupling the Active Ester Label of Ex. 1, or Thiol-Modified Oligonucleotides for Anchoring on Gold as Ligate Nucleic Acid Oligomers

The synthesis of the oligonucleotides takes place in an automatic oligonucleotide synthesizer (Expedite 8909; ABI 384 DNA/RNA Synthesizer) according to the synthesis protocol recommended by the manufacturer for a 1.0 μmol synthesis.

By default, the synthesis of the (signal) ligands takes place on A-CPG as the support material. Modifications at the 5′-position of the oligonucleotides take place with a coupling step prolonged to 5 minutes. The amino modifier C2 dT (Glen Research 10-1037) is incorporated into the sequences with the relevant standard protocol.

The constitution of 3′-thiol-modified ligate oligonucleotides (or HO—(CH₂)₂—SS—CH₂)₂OPO₃ oligonucleotides) takes place on 1-O-dimethoxytrityl-propyl-disulfide-CPG support (Glen Research 20-2933) analogously to standard protocols, the oxidation steps being carried out with a 0.02 M iodine solution to avoid oxidative cleavage of the disulfide bridge.

During synthesis, the coupling efficiencies are determined online photometrically or conductometrically via the DMT cation concentration.

The oligonucleotides are deprotected with concentrated ammonia (30%) at 37° C. over a period of 16 h. The purification of the oligonucleotides takes place by means of RP-HPL chromatography according to standard protocols (solvent system: 0.1 M triethylammonium acetate buffer, acetonitrile), the characterization by MALDI-TOF MS.

### Example 3

### Converting the Amino-Modified Oligonucleotides (Ex. 2) with the N-hydroxy Active Esters (Ex. 1)

The amino-modified oligonucleotides are dissolved in 0.1 M borate buffer (pH 8.5) and converted with the N-hydroxysuccinimide active esters dissolved in DMSO according to the protocol from Molecular Probes (Labeling Amine-Modified Oligonucleotides). The purification of the oligonucleotides takes place by means of RP-HPL chromatography according to standard protocols (solvent system: 0.1 M triethylammonium acetate buffer, acetonitrile), the characterization by MALDI-TOF MS.

### EXAMPLE 4

### Producing the Oligonucleotide Electrode Au—S(CH₂)₂-ss-oligo

Au—S(CH₂)₂-ss-oligo is produced in 2 steps, namely constituting the conductive surface and derivatizing the surface with the ligate oligonucleotide in the presence of a suitable monofunctional linker (incubation step).

An approx. 100 nm thin gold film on mica (muscovite lamina) forms the support material for the covalent attachment of the double-strand oligonucleotides. For this purpose, freshly cleaved mica is purified with an argon-ion plasma in an electrical discharge chamber and gold (99.99%) is applied by electrical discharge in a layer thickness of approx. 100 nm. Thereafter, the gold film is freed of surface impurities (oxidation of organic accumulations) with 30% H₂O₂/70% H₂SO₄ and immersed in ethanol for approx. 20 minutes to dispel any oxygen adsorbed on the surface. After rinsing the surface with bidistilled water, a previously prepared 1×10⁻⁴ molar solution of the (modified) oligonucleotide is applied onto the horizontally mounted surface, such that the entire gold surface is wetted (incubation step, see also below).

For incubation, a modified 20-bp single-strand oligonucleotide having the sequence 5′-TAG CGG ATA ACA CAG TCA CC-3′ is used, which is esterified with (HO—(CH₂)₂—S)₂ at the phosphate group of the 3′-end to form P—O—(CH₂)₂—S—S—(CH₂)₂—OH (cf. ex. 2). An approx. 10⁻⁵ to 10⁻¹ molar propanethiol solution (or another thiol or disulfide of suitable chain length) is added to a 5×10⁻⁵ molar solution of this oligonucleotide in HEPES buffer (0.1 molar in water, pH 7.5 with 0.7 molar addition of TEA TFB), the gold surface of a test site completely wetted and incubated for 2-24 hours. During this reaction time, the disulfide spacer P—O—CH₂)₂—S—S—(CH₂)₂—OH of the oligonucleotide is homolytically cleaved. In this process, the spacer forms a covalent Au—S bond with Au atoms of the surface, thus causing a 1:1 coadsorption of the ss-oligonucleotide and the cleaved 2-hydroxy-mercaptoethanol. The free propanethiol that is also present in the incubation solution is likewise coadsorbed by forming an Au—S bond (incubation step). Instead of the single-strand oligonucleotide, this single-strand may also be hybridized with its unmodified complementary strand.

### EXAMPLE 5

### Alternative Production of the Oligonucleotide Electrode Au—S(CH₂)₂-ss-oligo

Au—S(CH₂)₂-ss-oligo is alternatively produced in 3 steps, namely constituting the conductive surface, derivatizing the surface with the ligate oligonucleotide (incubation step) and postcoating the electrode modified in this way with a suitable monofunctional linker (postcoating step).

An approx. 100 nm thin gold film on mica (muscovite lamina) forms the support material for the covalent attachment of the ligate oligonucleotides, cf. ex. 4.

For incubation, a modified 20-bp single-strand oligonucleotide having the sequence 5′-TAG CGG ATA ACA CAG TCA CC-3′ is used, which is esterified with (HO—(CH₂)₂—S)₂ at the phosphate group of the 3′-end to form P—O—(CH₂)₂—S—S—(CH₂)₂—OH. The gold surface of a test site is wetted with an approx. 5×10⁻⁵ molar solution of this oligonucleotide in HEPES buffer (0.1 molar in water, pH 7.5) and incubated for 2-24 hours. During this reaction time, the disulfide spacer P—O—(CH₂)₂—S—S—(CH₂)—OH of the oligonucleotide is homolytically cleaved. In this process, the spacer forms a covalent Au—S bond with Au atoms of the surface, thus causing a coadsorption of the ss-oligonucleotide and the cleaved 2-hydroxy-mercaptoethanol (incubation step).

Thereafter, the gold electrode modified in this way is completely wetted with an approx. 10⁻⁵ to 10⁻¹ molar propanethiol solution (in water or buffer, pH 7-7.5) or with another thiol or disulfide (of suitable chain length) and incubated for 2-24 hours. After the incubation step, the free propanethiol coats remaining free gold surface by forming an Au—S bond.

### EXAMPLE 6

### Chronocoulometric Measurement in the Au-ss-oligo/Ferrocene-Modified Nucleic Acid Tetramer System in the Absence and Presence of Nucleic Acid Oligomer Ligands (Complementary to ss-oligo in Au-ss-oligo)

A probe electrode is produced according to ex. 5. For this purpose, the above-described HO—(CH₂)₂—SS—(CH₂)₂-modified oligonucleotide (sequence TAG CGG ATA ACA CAG TCA CC) is immobilized on gold (50 μmol oligonucleotide in phosphate buffer (500 mM K₂HPO₄/KH₂PO₄ pH 7), postcoating with 1 mM propanethiol in water).

Following addition of complementary double ferrocene-labeled nucleic acid tetramers (10 μM), a potential jump experiment is carried out. The values obtained by chronocoulometric measurement are shown in FIG. 3, curve 1. Following addition of the complementary target (5 μM), the potential jump experiment is repeated. The values obtained from the chronocoulometric measurement repeated subsequently are shown in FIG. 3, curve 2.

The diameter of the gold electrode used measures 6 mm, i.e. a surface area of 0.28 cm² is available for the immobilization of the ligate nucleic acid oligomers. The integrals of curves 1 and 2 (FIG. 3) yield a difference of 70×10⁻⁸ C (0.7 μC). This value corresponds to 2.5 μC/cm² or 1.6×10¹³ electrons/cm². Assuming maximum coverage with ligate oligonucleotides, in other words coverage with 7×10¹² ligate oligonucleotides per cm², an average of at least approx. 2.2 electrons per ligate oligonucleotide were thus converted, i.e. 2.2 ferrocene labels were displaced by hybridization of the ligate nucleic acid oligomer with the nucleic acid oligomer ligand. Thus, on average, 1.1 tetramers were displaced from the ligate oligonucleotide.

1. A method for detection of ligate-ligand association events, comprising the steps:
a) providing a modified surface, the modification consisting in the attachment of at least one type of ligate,
b) providing signal ligands,
c) providing a sample having ligands,
d) bringing a defined quantity of the signal ligands into contact with the modified surface and bringing the sample into contact with the modified surface,
e) detecting the signal ligands,
f) comparing the values obtained in step e) with reference values.

2. The method according to claim 1, wherein in step d), bringing a defined quantity of the signal ligands into contact with the modified surface and bringing the sample into contact with the modified surface take place simultaneously.

3. The method according to claim 1, wherein in step d) bringing a defined quantity of the signal ligands into contact with the modified surface and bringing the sample into contact with the modified surface take place separately.

4. The method according to claim 3, wherein as step d), first the step
d₁) bringing a defined quantity of the signal ligands into contact with the modified surface
and thereafter the step
d₂) bringing the sample into contact with the modified surface is carried out.

5. The method according to claim 4, wherein after step d₁) and before step d₂) the step
d₃) detecting the signal ligands
is carried out and in step f) the values obtained in step e) are compared with the reference values obtained in step d₃)

6. The method according to claim 5, wherein after step d₃) and before step d₂) the step
d₄) washing the modified surface
is carried out, and after step d₂) and before step e) the step
d₅) bringing the signal ligands into contact with the modified surface, the identical defined quantity of signal ligands being used as in step d₁)
is carried out.

7. The method according to claim 6, wherein after step d₃) and before step d₄) the step
d₆) setting conditions or taking actions that lead to at least predominant dissociation of ligates and signal ligands
is carried out.

8. The method according to claim 3, wherein as step d), first the step
d₂) bringing the probe into contact with the modified surface
and thereafter the step
d₁) bringing a defined quantity of the signal ligands into contact with the modified surface
is carried out.

9. The method according to claim 8, wherein after step e) the steps
e₁) setting conditions or taking actions that lead to at least predominant dissociation of ligates and ligands,
e₂) washing the modified surface,
e₃) bringing the signal ligands into contact with the modified surface, the identical defined quantity of signal ligands being used as in step d₁),
e₄) detecting the signal ligands
are carried out and in step f), the values obtained in step e) are compared with the reference values obtained in step e₄).

10. The method according to claim 9, wherein in step d₆) or in step e₁), chaotropic salts are added.

11. The method according to any one of claims 1, 2, and 3, wherein as step a) the step a) providing a modified surface, the modification consisting in the attachment of at least two types of ligates, and the differing types of ligates being bound to the surface in spatially substantially separate regions is carried out, after step c) and before step d) the step c₁) adding a ligand to the sample, the ligand being a binding partner having a high association constant of a ligate that is bound to the surface in a specific region T₁₀₀, the ligand being added in a quantity that is greater than the quantity of ligands needed to completely associate the ligates of the T₁₀₀ test sites is carried out and in step f), the values obtained in step e) are compared with the value obtained for the T₁₀₀ region.

12. The method according to claim 11, wherein as step a) the step
a) providing a modified surface, the modification consisting in the attachment of at least three types of ligates, and the differing types of ligates are bound to the surface in spatially substantially separate regions, at least one type of ligate being attached to the surface in a specific region T₀, and no binding partner having a high association constant to this ligate is contained in the probe
is carried out and in step f) the values obtained in step e) are compared with the value obtained for the T₁₀₀ region and with the value obtained for the T₀ region.

13. The method according to claim 12, wherein before step d) the step c₂) adding at least one additional type of ligand to the sample, the ligand in the sample provided in step c) not being contained and the ligand exhibiting an association constant >0 to a ligate that is bound to the surface in a specific region Tₙ, the ligand being added in a quantity such that, after step d), n% of the ligates in the Tₙ region are present in associated form is carried out and in step f), the values obtained in step e) are compared with the value obtained for the T₁₀₀ region, with the value obtained for the T₀ region and with the values obtained for the Tₙ regions.

14. The method according to claim 13, wherein the signal ligands are added in a quantity that is greater than the quantity of signal ligands needed to completely associate the ligates of the T₁₀₀ test sites.

15. The method according to claim 14, wherein the signal ligands are modified with a detection label.

16. The method according to claim 14, wherein the signal ligands are modified with multiple detection labels.

17. The method according to claim 16, wherein a fluorophore is used as the detection label, especially a fluorescent dye, especially Texas Red, a rhodamine dye or fluorescein.

18. The method according to claim 16, wherein a redox-active substance is used as the detection label.

19. The method according to claim 18, wherein riboflavin, a quinone, especially pyrrolloquinoline quinone, ubiquinone, anthraquinone, naphthoquinone, menaquinone, or derivatives thereof, a metallocene, especially a ferrocene or a cobaltocene, a metallocene derivative, especially a ferrocene derivative or a cobaltocene derivative, a porphyrin, methylene blue, daunomycin, a dopamine derivative, a hydroquinone derivative, especially a para- or ortho-dihydroxy-benzene derivative, a para- or ortho-dihydroxy-anthraquinone derivative or a para- or ortho-dihydroxy-naphthoquinone derivative is used as the redox-active substance.

20. The method according to claim 18, wherein a transition metal complex, especially a Cu, Fe, Ru, Os or Ti transition metal complex is used as the redox-active substance.

21. The method according to claim 20, wherein a transition metal complex having one or more ligands selected from the group consisting of pyridine, 4,7-dimethylphenanthroline, 9,10-phenanthrene quinonediimine, porphyrins and substituted porphyrin derivatives is used.

22. The method according to claim 21, wherein the modified surface is a conductive surface.

23. The method according to claim 22, wherein the detection of the signal ligands takes place through a surface-sensitive detection method.

24. The method according to claim 23, wherein the detection of the signal ligands takes place through a spectroscopic, an electrochemical or an electrochemiluminescent method.

25. The method according to claim 24, wherein the spectroscopic detection takes place through detecting the fluorescence, especially the total internal reflection fluorescence (TIRF), of the signal ligands.

26. The method according to claim 24, wherein the electrochemical detection takes place through amperometry or chronocoulometry.

27. The method according to claim 26, wherein substrates, cofactors or coenzymes are used as ligands, and proteins or enzymes are used as ligates.

28. The method according to claim 26, wherein antibodies are used as ligands, and antigens or antigen-antibody complexes are used as ligates.

29. The method according to claim 26, wherein antigens are used as ligands, and antibodies are used as ligates.

30. The method according to claim 26, wherein receptors are used as ligands, and hormones are used as ligates.

31. The method according to claim 26, wherein hormones are used as ligands, and receptors are used as ligates.

32. The method according to claim 26, wherein nucleic acid oligomers are used as ligands, and nucleic acid oligomers that are complementary thereto are used as ligates.

33. The method according to claim 32, wherein in step d₅) or in step e₁) the temperature is raised above the melting temperature of the ligate—signal ligand—oligonucleotide.

34. The method according to claim 33, wherein in step d₅) or in step e₁) a potential that lies above the electrostringent potential is applied.

35. The method according to claim 34, wherein the nucleic acid oligomers used as ligates comprise 3 to 70 or 3 to 50 bases, especially 8 to 50 or 5 to 30 bases, particularly preferably 10 to 30 or 8 to 25 bases.

36. A use of substrates, cofactors, coenzymes, proteins, enzymes, antibodies, antigens, receptors, hormones and nucleic acid oligomers as ligates and/or signal ligands and/or ligands in a method according to claim 35.

37. A kit for carrying out a method of detection of ligate-ligand association events, comprising a modified surface, the modification consisting in the attachment of at least one type of ligate; and an effective quantity of signal ligands.

38. The kit according to claim 37, wherein the kit additionally comprises reference values for comparison with the values obtained from the detection of the signal ligands.

39. The kit according to claim 37, wherein the modified surface comprises at least one T₀ region and at least one T₁₀₀ region.

40. The kit according to claim 39, wherein the modified surface additionally comprises at least one Tₙ region.


SEQUENCE LISTING (3 sequences)

SEQ ID NO: 1 - DNA, 20 bp, Artificial Sequence
  Chemically Synthesized
tagcggataacacagtcacc

SEQ ID NO: 2 - DNA, 20 bp, Artificial Sequence
  Chemically Synthesized
tagcggataacacagtcacc

SEQ ID NO: 3 - DNA, 20 bp, Artificial Sequence
  Chemically Synthesized
tagcggataacacagtcacc